SEQUENCE LISTING (18 sequences)

SEQ ID NO: 1 - protein, 533 aa, Adeno associated virus-2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 2 - protein, 5 aa, Artificial Sequence
  cytoplasmic dynein binding motif
KSTQT

SEQ ID NO: 3 - protein, 5 aa, Artificial Sequence
  cytoplasmic dynein binding motif
GIQVD

SEQ ID NO: 4 - protein, 5 aa, Artificial Sequence
  cytoplasmic dynein binding motif
SKCSR

SEQ ID NO: 5 - protein, 735 aa, Adeno associated virus -2 [sequence omitted]

SEQ ID NO: 6 - protein, 598 aa, Adeno associated virus -2
MAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - protein, 885 aa, Homo sapiens
MSTMRLLTLALLFSCSVARAACDPKIVNIGAVLSTRKHEQMFREAVNQANKRHGSWKIQLNATSVTHKPNAIQMALSVCEDLISSQVYAILVSHPPTPNDHFTPTPVSYTAGFYRIPVLGLTTRMSIYSDKSIHLSFLRTVPPYSHQSSVWFEMMRVYSWNHIILLVSDDHEGRAAQKRLETLLEERESKAEKVLQFDPGTKNVTALLMEAKELEARVIILSASEDDAATVYRAAAMLNMTGSGYVWLVGEREISGNALRYAPDGILGLQLINGKNESAHISDAVGVVAQAVHELLEKENITDPPRGCVGNTNIWKTGPLFKRVLMSSKYADGVTGRVEFNEDGDRKFANYSIMNLQNRKLVQVGIYNGTHVIPNDRKIIWPGGETEKPRGYQMSTRLKIVTIHQEPFVYVKPTLSDGTCKEEFTVNGDPVKKVICTGPNDTSPGSPRHTVPQCCYGFCIDLLIKLARTMNFTYEVHLVADGKFGTQERVNNSNKKEWNGMMGELLSGQADMIVAPLTINNERAQYIEFSKPFKYQGLTILVKKEIPRSTLDSFMQPFQSTLWLLVGLSVHVVAVMLYLLDRFSPFGRFKVNSEEEEEDALTLSSAMWFSWGVLLNSGIGEGAPRSFSARILGMVWAGFAMIIVASYTANLAAFLVLDRPEERITGINDPRLRNPSDKFIYATVKQSSVDIYFRRQVELSTMYRHMEKHNYESAAEAIQAVRDNKLHAFIWDSAVLEFEASQKCDLVTTGELFFRSGFGIGMRKDSPWKQNVSLSILKSHENGFMEDLDKTWVRYQECDSRSNAPATLTFENMAGVFMLVAGGIVAGIFLIFIEIAYKRHKDARRKQMQLAFAAVNVWRKNLQQYHPTDITGPLNLSDPSVSTVV

SEQ ID NO: 8 - protein, 1464 aa, Homo sapiens [sequence omitted]

SEQ ID NO: 9 - protein, 1484 aa, Homo sapiens
MKPRAECCSPKFWLVLAVLAVSGSRARSQKSPPSIGIAVILVGTSDEVAIKDAHEKDDFHHLSVVPRVELVAMNETDPKSIITRICDLMSDRKIQGVVFADDTDQEAIAQILDFISAQTLTPILGIHGGSSMIMADKDESSMFFQFGPSIEQQASVMLNIMEEYDWYIFSIVTTYFPGYQDFVNKIRSTIENSFVGWELEEVLLLDMSLDDGDSKIQNQLKKLQSPIILLYCTKEEATYIFEVANSVGLTGYGYTWIVPSLVAGDTDTVPAEFPTGLISVSYDEWDYGLPARVRDGIAIITTAASDMLSEHSFIPEPKSSCYNTHEKRIYQSNMLNRYLINVTFEGRNLSFSEDGYQMHPKLVIILLNKERKWERVGKWKDKSLQMKYYVWPRMCPETEEQEDDHLSIVTLEEAPFVIVESVDPLSGTCMRNTVPCQKRIVTENKTDEEPGYIKKCCKGFCIDILKKISKSVKFTYDLYLVTNGKHGKKINGTWNGMIGEVVMKRAYMAVGSLTINEERSEVVDFSVPFIETGISVMVSRSNGTVSPSAFLEPFSADVWVMMFVMLLIVSAVAVFVFEYFSPVGYNRCLADGREPGGPSFTIGKAIWLLWGLVFNNSVPVQNPKGTTSKIMVSVWAFFAVIFLASYTANLAAFMIQEEYVDQVSGLSDKKFQRPNDFSPPFRFGTVPNGSTERNIRNNYAEMHAYMGKFNQRGVDDALLSLKTGKLDAFIYDAAVLNYMAGRDEGCKLVTIGSGKVFASTGYGIAIQKDSGWKRQVDLAILQLFGDGEMEELEALWLTGICHNEKNEVMSSQLDIDNMAGVFYMLGAAMALSLITFICEHLFYWQFRHCFMGVCSGKPGMVFSISRGIYSCIHGVAIEERQSVMNSPTATMNNTHSNILRLLRTAKNMANLSGVNGSPQSALDFIRRESSVYDISEHRRSFTHSDCKSYNNPPCEENLFSDYISEVERTFGNLQLKDSNVYQDHYHHHHRPHSIGSASSIDGLYDCDNPPFTTQSRSISKKPLDIGLPSSKHSQLSDLYGKFSFKSDRYSGHDDLIRSDVSDISTHTVTYGNIEGNAAKRRKQQYKDSLKKRPASAKSRREFDEIELAYRRRPPRSPDHKRYFRDKEGLRDFYLDQFRTKENSPHWEHVDLTDIYKERSDDFKRDSISGGGPCTNRSHIKHGTGDKHGVVSGVPAPWEKNLTNVEWEDRSGGNFCRSCPSKLHNYSTTVTGQNSGRQACIRCEACKKAGNLYDISEDNSLQELDQPAAPVAVTSNASTTKYPQSPTNSKAQKKNRNKLRRQHSYDTFVDLQKEEAALAPRSVSLKDKGRFMDGSPYAHMFEMSAGESTFANNKSSVPTAGHHHHNNPGGGYMLSKSLYPDRVTQNPFIPTFGDDQCLLHGSKSYFFRQPTVAGASKARPDFRALVTNKPVVSALHGAVPARFQKDICIGNQSNPCVPNNKNPRAFNGSSNGHVYEKLSSIESDV

SEQ ID NO: 10 - protein, 15 aa, Bos taurus
MNYALKGQGRTLYGF

SEQ ID NO: 11 - protein, 15 aa, Artificial Sequence
  Histogranin variant
SNYALKGQGRTLYGF

SEQ ID NO: 12 - protein, 116 aa, Homo sapiens
  misc_feature (81)..(81): Xaa can be any naturally occurring amino acid
MAAPAGRCLAPPQETVAVSQPPSPWVTMCDQKAVIKNADMSEETQQNSVECAPQALEKYNKERNTVAHIKKECDKKYNPTXHCIVGRNFSSYVTHETKHFIYFYLGQVAILLFKSG

SEQ ID NO: 13 - DNA, 48 bp, Artificial Sequence
  Primer
ggccgcactttgtacggattcggcttgtcgagacaagcagctaccgca

SEQ ID NO: 14 - DNA, 48 bp, Artificial Sequence
  Primer
ttggcccttaagtgtatagttcataccggtgttgcctctctggaggtt

SEQ ID NO: 15 - DNA, 60 bp, Artificial Sequence
  Primer
aagggacagggacgcactttgtacggattcgggttgtcagcagctaccgcagatgtcaac

SEQ ID NO: 16 - DNA, 42 bp, Artificial Sequence
  Primer
aagtgtatagttcgatccggtgaggttggtagatacagaacc

SEQ ID NO: 17 - DNA, 45 bp, Artificial Sequence
  Primer
actcagactactagtgggttgtcagcagctaccgcagatgtcaac

SEQ ID NO: 18 - DNA, 39 bp, Artificial Sequence
  Primer
cgacttatcttctccggtgaggttggtagatacagaacc

(19) United States
(12) Patent Application Publication
(10) Pub. No.: US 2009/0215870 A1
(43) Pub. Date: Aug. 27, 2009

(54) Compositions and methods for targeting of viral vectors

(76) Inventors: Ernest F. Terwilliger, Boston, MA (US); Jianfeng Xu, Newton, MA (US)

Correspondence Address: CLARK & ELBING LLP, 101 FEDERAL STREET, BOSTON, MA, 02110, US

(21) Appl. No.: 11/919,461

(22) PCT Filed: May 1, 2006
(86) PCT No.: PCT/US2006/016533

§ 371 Date: Dec. 16, 2008

Related U.S. Application Data
(60) Provisional application No. 60/676,032, filed on Apr. 29, 2005.

(51) Int. Cl. A61K 48/00; C07K 14/005; C07H 21/04; C12N 15/63; C12N 7/01; A61P 25/00

(52) U.S. Cl. 514/44 R; 530/350; 536/23.72; 435/320.1; 435/235.1

(57) ABSTRACT

Viral capsid proteins with increased targeting to neurons and increased retrograde transport are described. Chimeric virus particles comprising capsid proteins that (i) increase targeting to neurons and (ii) increase retrograde transport of the virus particle are described. Methods for introducing a nucleic acid into a neuron are also described.

### BACKGROUND OF THE INVENTION

The present invention relates to modified viral proteins, chimeric viral particles, and methods for targeting viral vectors to neurons.

There is keen interest in developing practical strategies for gene delivery into the brain and central nervous system (CNS). In addition to the many applications for basic research, the capability of safely and efficiently engineering CNS cell populations to express desired genes offers strong new therapeutic possibilities for numerous disorders, both chronic and acute. The distinctive features of neurons, and their complex interconnections with each other as well as with other CNS lineages impose stringent requirements upon any gene delivery system, rendering most vectors poorly suited for this purpose. Most gene vectors cannot penetrate the tegument surrounding the spinal cord, and direct injection into the cord tends, at best, to produce spots of localized intense gene transfer, with minimal lateral diffusion. While advances have been made been made using osmotic pumps to infuse vectors through the cerebrospinal fluid CSF and effective transfer can sometimes be achieved even with relatively inefficient gene vehicles, this highly invasive method is less than ideal.

Thus there is a need for more effective, less invasive, and safer strategies for targeting genes to neurons.

### SUMMARY OF THE INVENTION

The invention features modified capsid proteins potentially useful in viral vectors, chimeric viral particles, and methods for introducing such viral particles into neurons and other cells.

The invention features a modified capsid protein including a modification that increases binding of a viral particle including the capsid protein to an NMDA receptor relative to the binding of a viral particle not including the capsid protein to the NMDA receptor, where the modification is sufficient to increase the binding of a viral particle including the capsid protein to a neuron including the NMDA receptor. The modification may be an insertion of histogranin or a fragment thereof into the capsid protein, for example, an AAV capsid protein. The AAV capsid protein may be a VP3 capsid protein. The insertion into the VP3 capsid protein may be between amino acids 583 and 590 of the VP3 capsid protein. The invention may provide a viral vector including the modified capsid protein. Additionally, the modified capsid protein may further include a deletion of amino acid sequence from the capsid protein. The capsid protein including the deletion may be an AAV capsid protein, for example, a VP3 capsid protein (e.g., a VP3 capsid protein including a deletion of residues 584-589). The capsid protein may further include a modification that substantially decreases binding of a viral particle including the capsid protein to a heparin sulfate proteoglycan relative to the binding of a viral particle not including the capsid protein to the heparin sulfate proteoglycan, where the modification is sufficient to decrease binding of a viral particle including the capsid protein to a cell including the heparin sulfate proteoglycan (e.g. when the capsid protein includes at least 5% of capsid proteins present in the viral particle). The invention also includes a polynucleotide, for example, a vector, encoding the modified capsid protein (e.g., any of the above-described capsid proteins). The vector may be a viral vector (e.g., an AAV vector).

In another aspect, the invention provides a modified capsid protein including a modification, for example, an insertion of a cytoplasmic dynein binding motif (e.g., KSTQT (SEQ ID NO:2), GIQVD (SEQ ID NO:3), and SKCSR (SEQ ID NO:4) into the capsid protein) that increases binding of a viral particle including the capsid protein to a component of the cytoplasmic dynein complex, relative to the binding of a viral particle not including the capsid protein to the component of the cytoplasmic dynein complex, where the modification is sufficient to enhance retrograde transport of a viral particle including the capsid protein. The capsid protein may further include a modification that substantially decreases binding of a viral particle including the capsid protein to a heparin sulfate proteoglycan relative to the binding of a viral particle not including the capsid protein to the heparin sulfate proteoglycan, where the modification is sufficient to decrease binding of a viral particle including the capsid protein to a cell including the heparin sulfate proteoglycan (e.g., the capsid protein includes at least 5% of capsid proteins present in the viral particle). The capsid protein may be an AAV capsid protein (e.g., a VP3 capsid protein including a mutation or deletion of one or more of the following amino acid residues: R484, R487, R585, R588, and K532). The invention further features a polynucleotide encoding the modified capsid protein. The invention also features a viral vector (e.g., an AAV viral vector) including the polynucleotide.

In a third aspect, the invention provides a chimeric viral particle (e.g., an AAV viral particle) with (i) increased binding to a neuron including an NMDA receptor, and (ii) enhanced retrograde transport along a neuronal axon. The viral particle includes at least one modified capsid protein, the modified capsid protein including one of (1) a modification that increases binding of the viral particle to an NMDA receptor (e.g., an insertion including histogranin or a fragment thereof), and (2) a modification that increases binding of the viral particle to the cytoplasmic dynein complex, for example, an insertion of a cytoplasmic dynein binding motif (e.g., KSTQT (SEQ ID NO:2), GIQVD (SEQ ID NO:3), and SKCSR (SEQ ID NO:4)), wherein the viral particle has at least one of (a) increased binding to a neuron including an NMDA receptor and (b) increased retrograde transport when the viral particle contacts a neuron.

In a fourth aspect, the invention features a method of introducing a nucleic acid into a neuron, for example, a neuron in a subject (e.g., a human). The method includes administration of a viral vector including a modified capsid protein, the modified capsid protein including a modification that increases binding to an NMDA receptor relative to the binding of a capsid protein lacking the modification to the NMDA receptor, wherein the modification is sufficient to increase the binding of a viral vector including the modified capsid protein to a neuron including the NMDA receptor. The method may allow enhanced expression of the nucleic acid in a neuron of the subject relative to a viral vector lacking the modified capsid protein.

In a fifth aspect, the invention features a method of introducing a nucleic acid into a cell, for example a cell in a subject (e.g., a human). The method includes administration of a viral vector including a modified capsid protein, the modified capsid protein including a modification that increases binding of the modified capsid protein to the cytoplasmic dynein complex relative to the binding of a capsid protein lacking the modification to the cytoplasmic dynein complex, wherein the modification is sufficient to enhance retrograde transport in a cell of a viral vector including the modified capsid protein. The method may allow enhanced expression of the nucleic acid in a cell of the subject relative to a viral vector lacking the modified capsid protein.

By “capsid protein” is meant any viral structural protein, such as a structural protein of an adeno-associated virus (e.g., AAV-2). Exemplary capsid proteins include VP1 (SEQ ID NO:5), VP2 (SEQ ID NO:6), and VP3 (SEQ ID NO: 1) proteins encoded by the cap gene of AAV-2. Proteins substantially identical to these proteins or encoded by a polynucleotide that hybridizes to a polynucleotide encoding VP1 (SEQ ID NO:5), VP2 (SEQ ID NO:6), or VP3 (SEQ ID NO: 1) are also capsid proteins of the invention.

By “substantially identical” is meant a polypeptide or polynucleotide molecule exhibiting at least 25% identity to a reference amino acid sequence (for example, any one of the amino acid sequences described herein) or nucleic acid sequence (for example, any one of the nucleic acid sequences described herein). Preferably, such a sequence is at least 30%, 35%, 40%, 45%, 50%, 55%, 60%, or 65% identical, more preferably 70%, 75%, or over 80% identical, and most preferably 90%, 91%, 92%, 93%, 94%, or even 95%, 96%, 97%, 98%, or 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e.sup.-3 and e.sup.-100 indicating a closely related sequence.

By “hybridize” is meant pair to form a double-stranded complex containing complementary paired nucleic acid sequences, or portions thereof, under various conditions of stringency. (See, e.g., Wahl. and Berger, Methods Enzymol 152:399 (1987); Kimmel, Methods Enzymol 152:507 (1987)) For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180 (1977)); Grunstein and Hogness (Proc Natl Acad Sci USA 72:3961 (1975)); Ausubel et al. (Current Protocols in Molecular Biology, Wiley Interscience, New York (2001)); Berger and Kimmel (Guide to Molecular Cloning Techniques, Academic Press, New York, (1987)); and Sambrook et al. (Molecular Cloning: A Laboratory Manual, Cold Spring Harbor Laboratory Press, New York). Preferably, hybridization occurs under physiological conditions. Typically, complementary nucleobases hybridize via hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “modified capsid protein” is meant a capsid protein comprising one or more changes to its amino acid sequence (e.g., insertion, deletion, and substitution) or any post-translational modification (e.g., glycosylation, methylation, phosphorylation, and farnesylation).

By “VP3 capsid protein” is meant the protein with the sequence of SEQ ID NO: 1, a protein substantially identical to SEQ ID NO:1, or a protein encoded by a polynucleotide that hybridizes to a polynucleotide encoding SEQ ID NO: 1. Preferable VP3 capsid proteins are capable of assembling into a virus particle.

By “viral particle” is meant an assembly of viral capsid proteins and genetic material. The viral particle may be, for example, an AAV particle preferably comprising about 60 capsid proteins in a ratio of VP1:VP2:VP3 of about 1:1:18. AAV particles can be prepared, for example, as described herein.

By “chimeric viral particle” is meant a viral particle that includes a plurality of any one capsid protein (e.g., VP1, VP2, and VP3) such that at least one modified capsid protein is present in the viral particle. In some embodiments, the chimeric viral particle, for example, a chimeric AAV viral particle, may include two or more different modified capsid proteins (e.g., two VP3 capsid proteins, the first containing a modification that increases binding to an NMDA receptor, the second containing a modification that increases binding to the cytoplasmic dynein complex).

By “viral vector” is meant an viral particle (e.g., an AAV particle) that carries a polynucleotide for delivery to a cell. In one embodiment, recombinant AAV (rAAV) may carry no viral coding sequences, and thus no viral products are synthesized in the target cells.

By “modification” is meant any change to an amino acid sequence (e.g., insertion, deletion, and substitution) or post-translational modification to the amino acid sequence (e.g., glycosylation, methylation, phosphorylation, and farnesylation). A protein comprising a modification is “modified.”

By “fragment” is meant a chain of at least 4, 5, 6, 8, 10, 15, 20, or 25 amino acids or nucleotides which comprises any portion of a larger peptide or polynucleotide.

By “increases” or “enhances” is meant positively changing (e.g., increasing the binding affinity of a modified protein or viral particle containing the modified protein to a receptor) by at least 5%, more desirably at least 10%, 25%, or 50%, and even more desirably 100%, 200%, 500%, or more, relative to a control (e.g., the binding affinity of the wild-type protein or viral particle containing the wild-type protein to the receptor).

By “substantially decreases” is meant reducing (e.g., reducing the binding affinity of a modified protein or viral particle containing the modified protein to a receptor) by at least 5%, more desirably by at least 10%, 25%, or even 50%, relative to a control (e.g., the binding affinity of the wild-type protein or viral particle containing the wild-type protein to the receptor).

By “NMDA receptor” is meant a cell surface protein or group of proteins (e.g., subunits of an ion channel) that bind N-methyl-D-aspartic acid, glutamate, and glycine. NMDA receptors preferably comprise extracellular region(s) capable of binding agonists and transmembrane domains which form an ion (e.g., sodium, calcium, or potassium) channel. Exemplary NMDA receptors (e.g., subunits that form NMDA receptors) include the protein coded by the sequence of human NMDAR1 (SEQ ID NO:7), Human NMDAR2A (SEQ ID NO:8), and human NMDAR2B (SEQ ID NO:9).

By “histogranin” is meant a peptide of SEQ ID NO: 10 (see FIG. 9), or an NMDA receptor-binding variant (e.g., an agonist or antagonist) thereof (e.g., [Ser¹]HN (SEQ ID NO: 11)).

By “heparin sulfate proteoglycan” is meant a protein that comprises a post-translational modification by attachment of polysaccharide glycosaminoglycan moieties of repeating disaccharide units with various degrees of sulfation. Heparin sulfate proteoglycans are found on the surface of many cell types; AAV vectors may enter cells through binding to this protein.

By “cytoplasmic dynein complex” is meant a group of proteins comprising motor proteins involved in intracellular transport (e.g., retrograde transport), nuclear migration, and the orientation of the cell spindle at mitosis. Such proteins may be involved in vesicular transport along microtubules (e.g., along the length of axons). Exemplary cytoplasmic dynein complex proteins include the human cytoplasmic dynein 8 kD light chain (LC8) (SEQ ID NO:12).

By “cytoplasmic dynein binding motif” is meant any compound (e.g., an amino acid sequence) that specifically binds to an component of the cytoplasmic dynein complex. Exemplary cytoplasmic dynein binding motifs include KSTQT (SEQ ID NO:2), GIQVD (SEQ ID NO:3), and SKCSR (SEQ ID NO:4).

Other features and advantages of the invention will be apparent from the following Detailed Description, the drawings, and the claims.

### BRIEF DESCRIPTION OF THE DRAWINGS

FIG. 1 is an illustration of mutations introduced in the VP3 sequence in separate clones of plasmid pXX2, encoding the AAV-2 rep and cap functions.

FIGS. 2A-2E are images showing transduction of differentiated PC-12s cells with mutant AAV-2 vectors. After treatment with NGF for 7 days, differentiated PC-12s reached a final density of approximately 10⁶ cells/well. The cells were then either mock transduced (FIG. 2A) or received 10 μl of either standard rAAV-lacZ (FIG. 2B) or an engineered vector with the indicated capsid insert (FIGS. 2C-2E). The cells were fixed and stained with X-gal 48 hours later.

FIGS. 3A and 3B are images showing inhibition of NMDA-R dependent vector uptake and DMC dependent transport in PC-12 cells. FIG. 3A shows PC-12 cells pre-incubated with 20 micromolar histogranin peptide for 15 min before the addition of 10 μl of the AAV-HN1/DMC1 chimera carrying a lacZ transgene (right panel). Control cultures received AAV with no peptide (left panel). The medium was changed after 18 hours and the cells were fixed and stained with X-gal 2 days later as described in FIG. 2. FIG. 3B shows PC-12 cells pretreated with 20 μM sodium orthovanadate (Na₃VO₄) for 2 hours prior to addition of AAV-HN1/DMC1 (right panel). Control cultures received AAV with no vanadate (left panel). The medium was changed 14 hours later and the cells were fixed and stained as in FIG. 3A.

FIG. 4A-4E are images showing gene transfer into dissociated dorsal root ganglia (DRG) cultures using mutant AAV vectors. 2×10⁵ neurons/well were plated initially, and cultured for 7 days. The cells were then either mock transduced (FIG. 4A) or received 10 μl of the standard rAAV-lacZ (FIG. 4B) or one of the modified lacZ vectors (FIG. 4C-4E), as indicated. The cells were fixed and stained 48 hours after transduction.

FIG. 5A is a diagram showing the Campenot format. Two side chambers separated from a central well were established using Teflon dividers attached with grease to a 35 mm dish. 10⁵ neurons were plated initially in each central chamber. Guided by an NGF gradient between the central and side chambers, axons extend into the side chambers along parallel scratches etched in the plastic. Cell survival after 8 days was estimated at 50 percent.

FIG. 5B is a set of images showing AAV-mediated gene transfer into Campenot cultures. Eight days after establishment of the cultures in FIG. 5A, 10 μl of each vector was added to one side chamber of each culture. The other side chamber was left untreated. 48 hours later, the central and side chambers were fixed and stained for β-gal expression. Horizontal lines visible across the central chambers are grooves etched in the plastic as a guide for neurite outgrowth.

FIG. 5C is a set of images showing histogranin (HN) inhibition of gene transfer mediated by a chimeric AAV vector. HN peptide was added to one axon chamber of different Campenot cultures at a final concentration of either 20 or 50 μM, 10 min prior to addition of 10 μl of AAVHN1/DMC1. After 18 hours, the medium was changed in this chamber and the culture maintained for an extra 30 hours before fixing and staining as described in FIG. 3B.

FIG. 6 is an image of a gel showing co-immunoprecipitation of LC8 light chain with an AAV capsid antibody. 293 cells were transfected with either the standard pXX2 or mutant pXX2-DMC1 plasmids. Untransfected cells served as a negative control. Twenty four hours later, clarified cell lysates were prepared under non-denaturing conditions and immunoprecipitated with an AAV capsid antibody (A20). The precipitated immune complexes (lanes 2 to 5) as well as their respective supernatants (lanes 5 to 9) were resolved by SDS-PAGE and immunoblotted with anti-LC8 antibody. Lane 1, input control lysate; lane 2, IP of control lysate; lane 3, IP of lysate from cells transfected with pXX2; lane 4, IP of lysate from cells transfected with pXX2-DMC1; lane 5, same as lane 4 except the anti-AAV antibody was omitted; lanes 6 to 9, supernatant from IPs corresponding to lanes 2 to 5, respectively; lane 10, protein G-agarose alone.

FIG. 7 is a set of images showing transduction of non-neuronal cells with standard rAAV-2 or AAV-DMC1. HeLa cells, rat astrocytes, or CEM cells were transduced with either of the two vectors, fixed, and stained with X-gal 48 hours later. An MOI of about 50 was used for the cell lines except CEM, where the MOI was 100.

FIG. 8 is a set of images showing transduction of 3T3 cells by standard rAAV-2 or AAV-DMC1. 3T3 cells were incubated in the presence or absence of 10 mM HU for 2 hours prior to rinsing with PBS and replenishment with new medium. The HU treated, as well as the untreated control cells, were then transduced with 10 μl of either the standard AAV-2 or AAV-DMC1 lacZ vectors. The cells were fixed and stained with X-gal 48 hours after transduction.

FIGS. 9A-9D are a list of sequences.

### DETAILED DESCRIPTION

There is strong interest in developing practical strategies for gene delivery into the brain and central nervous system (CNS). Direct delivery into the brain or spinal cord is highly invasive as well as inefficient and/or hazardous with most vector systems. Here, gene vehicles which are taken up effectively by axons and home to neuron cell bodies in the spinal cord or other locations following delivery to peripheral sites were generated. The ability to deliver therapeutic genes in such vehicles enables new and novel approaches to treating multiple neurological disorders. Vectors derived from adeno-associated virus (AAV), a harmless human parvovirus, are a starting point for such vehicles. Enhancing axonal uptake of AAV and conferring efficient retrograde transport capabilities upon the virus can produce a near ideal gene transfer vehicles for the CNS. To enhance retrograde transport of the virus, peptides mimicking consensus binding domains for cytoplasmic dynein were inserted into the capsid by directed mutagenesis. In separate clones, peptides derived from a well-characterized NMDA receptor antagonist, histogranin (HN), were introduced to give the capsid a specific affinity for this receptor. When combined, the two classes of functional changes enabled efficient gene transfer into neurons under conditions not permissive for standard AAV-2 vectors. These results hold strong promise for the development of convenient vehicles to target genes and other sequences to neurons.

A convenient, minimally invasive approach would enable the vector to be delivered by simple injection, such as IM or IV. Such are the natural routes of infection of many pathogenic neurotropic viruses, upon reaching the blood or epithelial linings (Leopold et al., 2000. Hum. Gene Ther. 1: 151-165; Jacob et al., 2000. J Virol. 74:10217-10222). While certain lentiviral and HSV vectors travel very efficiently by this route, they bring their own special concerns and issues such as safety (Mikkers and Berns, 2003. Adv. Cancer Res. 88:53-99; Fortunato and Spector, 2003. Rev. Med. Virol. 13:21-37; Pakzaban et al., 1994. Hum. Gene Ther. 5:987-995).

Recombinant gene vectors derived from AAV (rAAV) offer starting candidates for applications in the CNS. Derived from a family of small non-pathogenic human parvoviruses, AAV vectors are capable of efficiently delivering gene cassettes of up to about 5 Kb. rAAV carry no viral coding sequences, so no viral products are synthesized in the target cells. Unlike retroviruses, integration is not a prerequisite for transcription of AAV gene cassettes. Integration by standard AAV vectors, as opposed to the wild-type (wt) virus, is slow, inefficient, and non-specific, and the majority of transgenes persist as highly stable, actively transcribed episomes, minimizing concerns about insertional mutagenesis. As a consequence, recombinant AAV are poorly suited for long term gene transfer into rapidly dividing cells but persist readily for months to years in slowly dividing or non-dividing lineages. A further key advantage is the low immunogenicity of the AAV capsid compared with other viral vectors such as adenovirus (Bessis et al., 2004. Gene Ther. 11 (Suppl 1):S10-S17). Although some variability has been reported with strain of animal and site of administration, exposure to an rAAV typically does not elicit a destructive cellular immune response against successfully transduced cells in immunocompetent animals.

Although AAV is a human virus, AAV vectors function equally effectively in cells of many other species, including rodents, dogs, and other primates, streamlining transitions from animal model systems to clinical trials. All these features serve to make rAAV increasingly popular as both research tools and for gene therapy applications, as rAAV gains increased acceptance for use in human gene therapy trials (Kay et al., 2000. Nat. Genet. 24:257-261; Athanasopoulos et al., 2000. Int. J. Mol. Med. 6:363-375; Mandel and Burger, 2004. Curr. Opin. Mol. Ther. 6:482-490).

The natural cellular tropism of AAV is broad, and standard AAV vectors have been used to transduce a spectrum of cell types, especially in vitro, including neurons. Unfortunately for applications in the CNS, standard vectors derived from AAV-2, the most commonly used and best characterized serotype, do not disperse widely after injection in the brain and are limited in their capacity for retrograde transport. Consequently they do not reach neuron cell bodies efficiently after injection into peripheral sites. For example, because of the high affinity of AAV for myocytes, AAV is principally absorbed into muscle fibers after IM injection. At least two studies failed to find the vector in spinal cord neurons following IM injection of AAV-2 vectors (Martinov et al., 2002. Anat. Embryol. 205:215-221; Wang et al., 2002. J. Neurosci. 22:6920-6928). Deficient retrograde transport in brain neurons of AAV has also been reported (Chamberlin et al., 1998. Brain Res. 793:169-175). Recently, Kaspar et al. (2003. Science 301:839-842) demonstrated retrograde transport of an AAV-2 IGF-1 vector to motor neurons after injection in SODG93A mice, sufficient for biological effects of the treatment to be apparent, but still at low levels that emphasize the overall inefficiency of the process. In addition to poor retrograde transport, despite the susceptibility of neurons to the virus, AAV may not be absorbed well by neural axons and dendrites.

Two distinct properties crucial to viruses targeting neurons appear lacking in standard AAV-2 (i) a specific means of entry and (ii) a mechanism for keying into an efficient retrograde transport pathway. Viruses exhibiting efficient uptake and retrograde transport along neural axons include the rabies viruses, some herpes viruses, some of the complex lentiviruses, and certain pathogenic parvoviruses. In some instances the features of their capsids which confer these properties have been identified, enabling attempts at reengineering such desirable properties into more innocuous vectors such as AAV. This is assisted by the availability of detailed X-ray crystallographic structures, particularly for the AAV-2 capsid (Xie et al., 2002. Proc. Natl. Acad. Sci. USA 99:10405-10410), as well as the identification of domains in the major capsid protein (VP3) tolerant of short peptide inserts. Some success has already been achieved altering the tropism of AAV-2 by inserting peptides in these domains (Shi et al., 2001. Hum. Gene Ther. 12:1697-1711; Wu et al., 2000. J. Virol. 74:8635-8647; Grifman et al., 2001. Mol. Ther. 3:964-975; Nicklin et al., 2001. Mol. Ther. 4:174-181).

Retargeting of a viral vector such as AAV (e.g., AAV-2) can be achieved by modification of the capsid proteins with sequences that increase binding to specific cellular receptors (e.g., the NMDA receptor). Standard AAV targeting (e.g., targeting to the heparin sulfate proteoglycan) can be decreased by deletion of sequences that bind to the wild-type target. Additional properties (e.g., increase in retrograde transport) can also be conferred on viral particles through similar modifications of capsid proteins.

In summary, the histogranin (HN) mutation, the DMC1 mutations, and their combination described herein represent a highly promising set of engineered gene vectors for targeting expression of genes contained in viral vectors to cells (e.g., neuronal cells). While additional refinements may be undertaken to further optimize their performance, the chimeric vector is able to deliver and express its gene successfully, in vitro as well as in vivo, under conditions in which the standard vector could not. The ability to deliver therapeutic sequences in vehicles specifically tailored to CNS populations can permit treatment of an array of serious debilitating neurological disorders.

The following examples are meant to illustrate the invention and should not be construed as limiting.

### Example 1

### Generation of Modified Capsid Proteins, Polynucleotides, and Capsids

The present invention includes modified capsid proteins and polynucleotides that encode modified capsid proteins (e.g., AAV capsid proteins) with increased binding to NMDA receptors and capsid proteins with increased binding to the cytoplasmic dynein motor complex (DMC). Modifications (e.g., insertions, deletions, or mutations) to capsid proteins of a viral vector such as AAV (e.g., AAV-2) can be generated using molecular biology techniques standard in the art (e.g., as described herein) to generate polynucleotide sequences that code for the desired modified protein. Such proteins and their encoding polynucleotides may further include deletions or mutations that decrease normal targeting (e.g., targeting to the heparin sulfate proteoglycan).

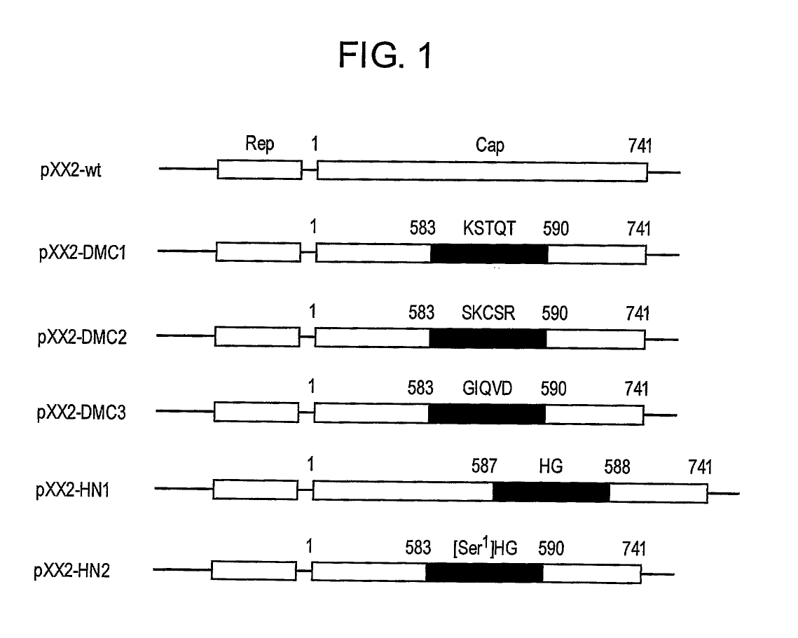

In the present example, the lacZ gene sequence was cloned into an AAV-based vector plasmid, pACP, which has been described previously (Cucchiarini et al., 2003. Gene Ther. 10:657-667). Mutagenesis of the AAV capsid was carried out using the ExSite PCR-based Site-directed Mutagenesis Kit (Stratagene, La Jolla, Calif.) using pXX2 (Xiao et al., 1998. J. Virol. 72:2224-2232) as the template plasmid. To target neurons specifically, NMDA receptor binding sequences such as HN (SEQ ID NO: 10; Lemaire et al., 1993. Eur. J. Pharmacol. 245:247-256; Lemaire et al., 1995. Life Sci. 56:1233-1241) were used. A short 15-amino acid peptide, HN is a potent NMDA receptor antagonist and efficiently displaces NMDA receptor ligand binding (Lemaire et al., 1993. Eur. J. Pharmacol. 245:247-256; Shukla et al., 1995. Pharmacol. Biochem. Behav. 50:49-54). The specificity of HN for NMDA receptors has been demonstrated by its ability to protect against NMDA induced convulsion, but not convulsion induced by other ionotropic glutamate receptor agonists such as AMPA or kainate (Lemaire et al., 1995. Life Sci, 56:1233-1241). For example, peptides mimicking either the natural HN sequence, [Met¹]HN, or an analog, [Ser¹]HN, with a single amino acid substitution that possesses a somewhat higher binding affinity and increased stability as a free peptide (Rogers and Lemaire, 1993) can be inserted in position 587 in VP3 (FIG. 1). Insertion of HN can be achieved, for example, by using the HN1 forward primer (SEQ ID NO:13) and the HN1 reverse primer (SEQ ID NO:14); or by using the HN2 forward primer (SEQ ID NO: 15) and the HN1 reverse primer (SEQ ID NO:16).

Generation of viral capsid proteins such as AAV capsid proteins (e.g., AAV-2) with increased binding to cytoplasmic dynein can be generated using the above-described methods by using primers designed to introduce motifs that increase binding the cytoplasmic dynein complex such as KSTQT (SEQ ID NO:2), GIQVD (SEQ ID NO:3), and SKCSR (SEQ ID NO:4) into a capsid protein (e.g., an AAV capsid protein). Many cellular proteins as well as neurotropic viruses (Mueller et al., 2002. J. Biol. Chem. 277:7897-7904; Topp et al., 1994. J. Neurosci. 14:318-325), rely upon cytoplasmic dynein, one of two major types of DMC, for retrograde transport (Schnapp et al., 1989. Proc. Natl. Acad. Sci. USA 86:1548-1552). Cytoplasmic dynein is a large protein complex composed of multiple subunits, with the heavy chains containing the motor domains, while intermediate and light chains (e.g., LC8 (SEQ ID NO: 12)) serve to bind the complex to different cargo proteins (Susalka et al., 2000. J. Neurocytol. 29:819-829; Pazour et al., 1998. J. Cell Biol. 141:979-992). Recent studies have identified specific components of light chains which are in direct association with different cargo proteins (Jacob et al., 2000. J. Virol. 74:10217-10222; Rodriguez-Crespo et al., 2001. FEBS Lett. 503:135-141; Mueller et al., 2002. J. Biol. Chem. 277:7897-7904). For example, using a pepscan technique, Rodriguez-Crespo et al. (supra) identified two consensus motifs, GIQVD and KSTQT, across a panel of 10 cargo proteins that all interacted with an 8 kDa light chain component (LC8). In particular, the KSTQT motif was common to proteins found in several neurotropic viruses, including Mokola virus, rabies virus, and African swine virus (Rodriguez-Crespo et al., supra). An SKCSR motif within the poliovirus receptor CD 155 was also shown to interact with a dynein light chain protein, Tctex-1 (Mueller et al., supra). None of these motifs are displayed on the standard AAV capsid. To enhance retrograde transport, peptides derived from several of these motifs were inserted into separate clones of VP3, once again at position 587. In one example, the construction of such a cap gene polynucleotide (named DMC1) with a KSTQT insert was generated using the DMC1 forward primer (SEQ ID NO: 17) and the DMC1 reverse primer (SEQ ID NO: 18). Capsid proteins with the other cytoplamsic dynein motifs described (SKCSR and GIQVD; termed DMC2 and DMC3 respectively) can be generated using appropriately designed primers.

For both NMDA- and cytoplasmic dynein-targeting modifications, regions complementary to the pXX2 template are included in each primer. In addition to the functional epitope, each insert may include flanking Thr-Gly and Gly-Leu-Ser residues 5′ and 3′ to the inserts, respectively (Shi et al., 2001. Hum. Gene Ther. 12:1697-1711) for flexibility. In these exemplary methods, a unique restriction site was included in each insert and/or deletion for screening purposes. All constructs were also verified by sequencing.

In both NMDA- and cytoplasmic dynein-targeting modifications, the insertions were placed near amino acid 587 of the VP3 capsid protein, located in loop IV of this protein, which is recognized as a tolerant site in the capsid, and is involved in the interaction of AAV-2 with heparin sulfate proteoglycan (HSPG) in the normal binding of the virus to the host cell (Girod et al., 1999. Nat. Med. 5:1052-1056; Grifman et al., 2001. Mol. Ther. 3:964-975; Shi et al., 2001. Hum. Gene Ther. 12:1697-1711; Ried et al., 2002. J. Virol. 76:4559-4566). Small disruptions in this domain can result in reduced HSPG binding, but need not interfere with virus assembly. In addition to the disruption of the native sequence at this site, a specific deletion can be introduced in most of the mutants, encompassing residues 584-589, including two arginines at 585 and 588. Two recent mutagenesis studies implicated these residues in the efficient interaction of the capsid with HSPG, although other sites (e.g., R484, R487, and K532) were also important (Opie et al., 2003. J. Virol. 77:6995-7006; Kern et al., 2003. J. Virol. 77:11072-11081). In the present invention, any of these sites may be used to decrease HSPG bind of the AAV capsid protein.

### Polypeptide Generation

Polypeptides of the invention can be generated from the above-described polynucleotides coding for such proteins using any methods standard in the art, such as those described below.

Viral Packing of Polypeptides

In a particularly useful embodiment of the invention, polynucleotides (e.g., polynucleotides encoding modified capsid proteins) can be expressed in a viral packaging system such as an rAAV system. Packaging of rAAV can be carried out according to protocols known in the art with some modifications (Xiao et al., 1998. J. Virol. 72:2224-2232). Briefly, vectors were packaged in a 3 plasmid system by co-complementation of the AAV vector plasmid with a second plasmid, pXX2 or one of its derivatives, encoding the AAV-2 replication and encapsidation functions, together with a third plasmid, pXX6 carrying essential adenoviral helper functions. Purification of the vector preparations can be achieved by a combination of passage over an iodixanol gradient followed by ion exchange chromatography using a 1- or 5-ml HiTrap Q column (Amersham Bioscience, Piscataway, N.J.) as is known in the art (Zolotukhin et al., 2002. Methods 28:158-167). rAAV vector stocks can be titered by real-time PCR using the ABI Prism 7700 Sequence Detection System from Perkin-Elmer Applied Biosystems (Foster City, Calif., USA), as described in Clark et al. (1999. Hum. Gene Ther. 10:1031-1039). rAAV doses can be calculated based on real-time PCR titers. Functional titers of rAAV vector preparations after purification are desirably on the order of 10¹⁰ per ml. MOI is defined as number of transgenes rather than virus particles.

Polypeptide Expression

In general, polypeptides for use in the invention may be produced by any standard technique, for example, by transformation of a suitable host cell with all or part of a polypeptide-encoding polynucleotide molecule or fragment thereof in a suitable expression vehicle.

Those skilled in the field of molecular biology will understand that any of a wide variety of expression systems may be used to provide the recombinant polypeptide. The precise host cell used is not critical to the invention. A polypeptide for use the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Current Protocols in Molecular Biology, Eds. Ausubel et al., John Wiley and Sons). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (Pouwels, P. H. et al., 1985, Supp. 1987).

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. As the gene encoding such a polypeptide is under the control of the T7 regulatory signals, expression of the polypeptide is achieved by inducing the expression of T7 RNA polymerase in the host cell. This is typically achieved using host strains which express T7 RNA polymerase in response to IPTG induction, Once produced, recombinant polypeptide is then isolated according to standard methods known in the art, for example, those described herein.

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The polypeptide of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, polypeptides expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide for use in the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant polypeptide can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides for use in the invention, particularly short peptide fragments, can also be produced by chemical synthesis (e.g., by the methods described in Solid Phase Peptide Synthesis, 2nd ed., 1984 The Pierce Chemical Co., Rockford, Ill.).

These general techniques of polypeptide expression and purification can also be used to produce and isolate useful peptide fragments or analogs (described herein).

### Example 2

### Generation of Chimeric Viral Particles

As distinct attributes, it is difficult to imagine a single small peptide insert conferring neuron specific uptake as well as efficient retrograde transport. However, the present invention demonstrates that new functional epitopes do not need to be reiterated in every capsid component for powerful effects to be achieved. Each AAV capsid is assembled from about 60 building blocks of VP3, as well as smaller amounts of other subunits (VP1 and VP2) produced by alternative splicing of the cap mRNA. Using the above-described rAAV packaging system with a mix of capsid gene plasmids (e.g., a first plasmid encoding a capsid protein with increased NMDA receptor binding and a second plasmid encoding a capsid protein with increased cytoplasmic dynein binding) incorporating different changes, when transfected together into packaging cells, results on average in many copies of each mutation expressed on the surface of each virus particle.

### Example 3

### Gene Transfer In Vitro Using Chimeric AAV Vectors

To test whether the chimeric AAV of the invention were taken up into neurons which then exhibited enhanced transgene expression, several in vitro systems were used. Synergistic effects of the mutations were seen in differentiated PC-12 cells and in cultures of dorsal root ganglia (DRG) neurons. PC-12 cells and DRG represent neuronal cell types poorly susceptible to AAV-2 mediated gene transfer. In the Campenot format, the latter also offered a rigorous test of the capacity of each vector for retrograde transport.

Gene Transfer into PC-12 Cells by Capsids with Engineered Peptide Motifs

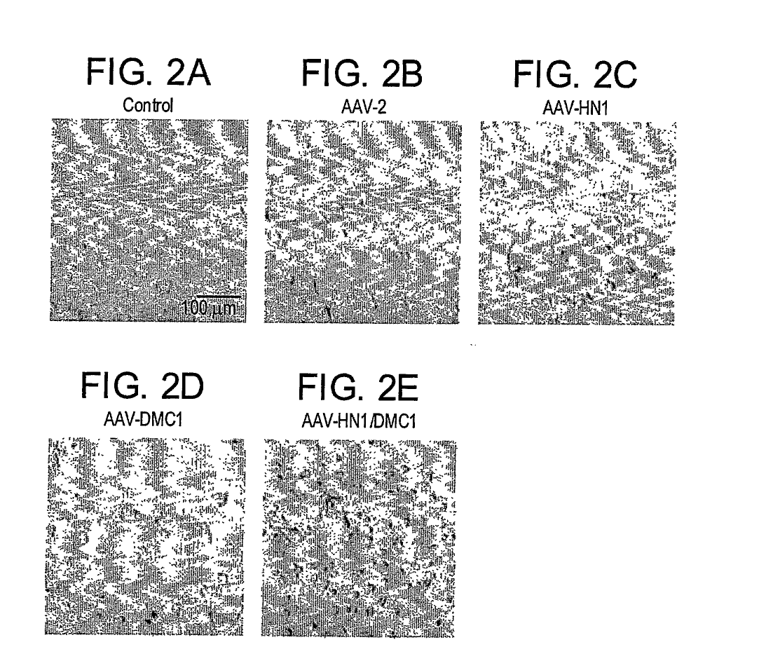

The properties of rAAV bearing these different peptides, alone or in combination, were compared to those of the unmodified AAV-2 vector in differentiated PC-12 cells, a rat pheochromocytoma line retaining many characteristic neuronal properties, including expression of NMDA receptors (Casado et al., 1996. J. Physiol. 490:391-404). In differentiated PC-12 cells, expression of the transgene following exposure to the standard rAAV-2 lacZ was poor (1-3 percent), consistent with previous experiences of our group and others. Modest, reproducible improvements in efficiency were seen with vectors bearing either the KSTQT motif (DMC1) or an HN peptide insert alone, resulting in up to several fold more lacZ expressing cells than the standard AAV-2, with up to 10-15 percent of the cells expressing β-galactosidase (β-Gal) at an MOI of about 100. However, after exposure to an equivalent dose of a chimeric vector bearing capsid proteins with either the DMC1 or HN1 motifs, the number of lacZ expressing cells was at least 6-8 fold higher again, with greater differences evident at lower input doses (FIG. 2). Comparisons between the two variants of the HN motif in PC-12 cells did not reveal significant phenotypic differences. Capsids bearing the DMC2 or DMC3 motifs did not display phenotypes distinct from AAV-2 and were not characterized further.

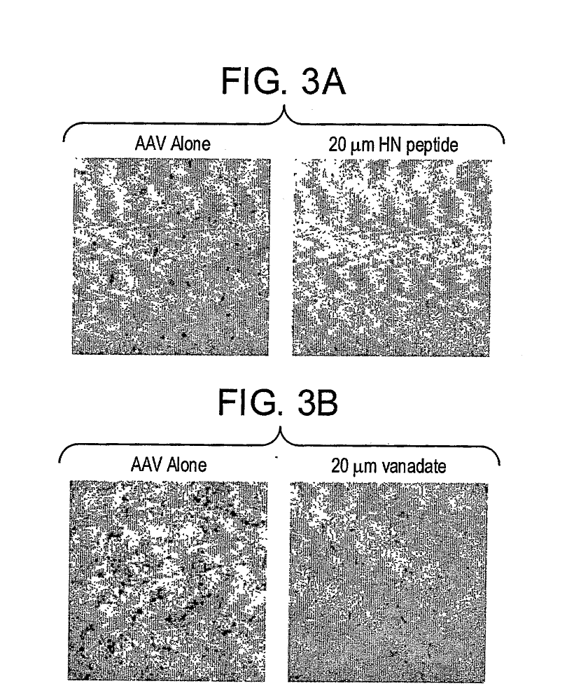

To confirm the enhanced gene expression by the double mutant was mediated at least in part via NMDA receptors, the transduction was competed with excess HN peptide (Bachem, Torrence, Calif.). Pre-incubation with HN reduced the transduction efficiency back to levels similar to those with standard AAV (FIG. 3A). Selected cultures were also pre-treated with sodium vanadate, a potent inhibitor of the dynein motor complex. This treatment also strongly suppressed gene transfer by the mutant vector, as shown in FIG. 3B, consistent with retrograde transport occurring via this pathway. A similar effect of vanadate upon gene transfer by standard rAAV-2 in susceptible cell lines such as 293 was not observed (not shown).

Gene Transfer into Dorsal Root Ganglia Neurons

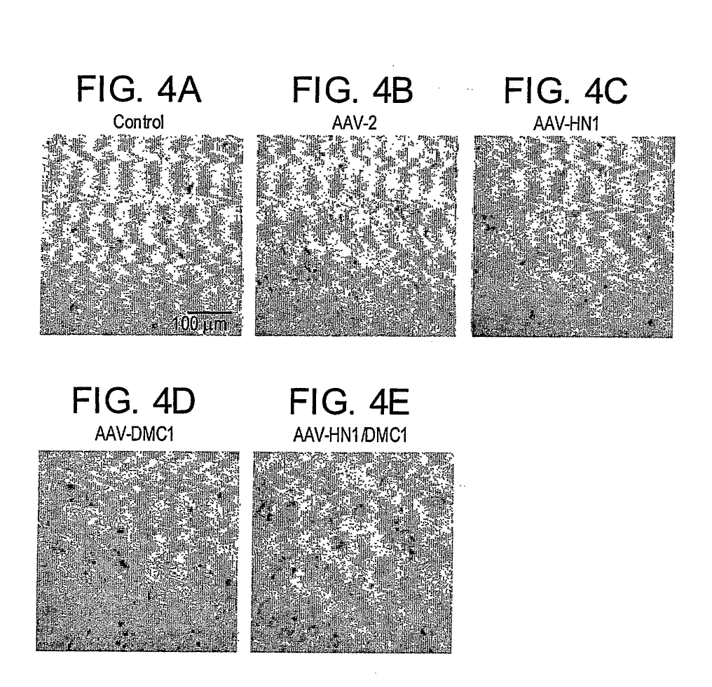

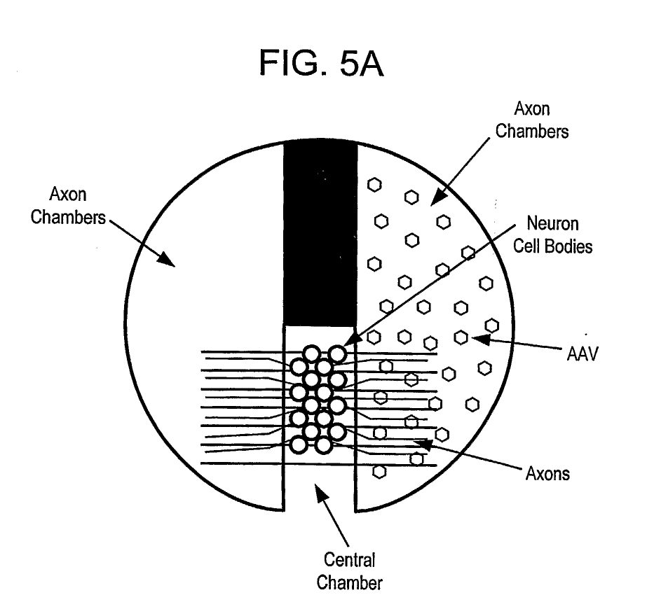

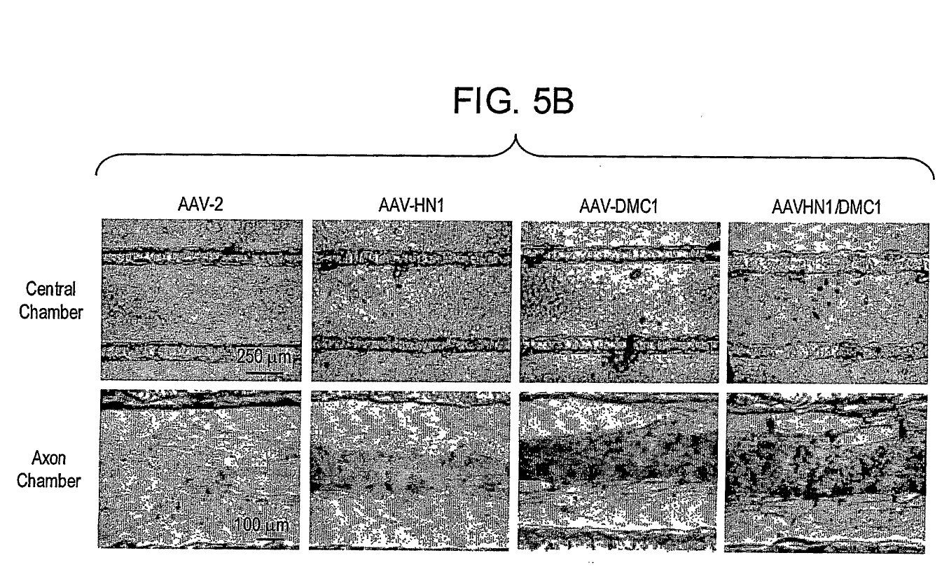

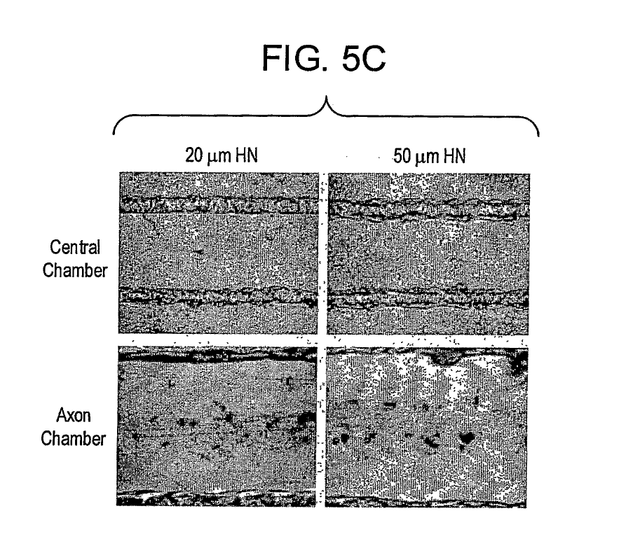

The AAV-DMC1 and AAV-HN1 viruses, as well as their chimeric combination, were next evaluated in cultures of sensory neurons isolated from neonatal rat dorsal root ganglia (DRG). Gene transfer by standard rAAV-2 in dissociated DRG was very poor, resulting in only a few percent of the cells expressing β-gal after exposure at high MOI. A vector bearing both the HN1 and DMC1 motifs transduced with much higher efficiency, with at least 50 percent of the cells expressing the transgene at a similar input dose. Vectors bearing single mutations yielded intermediate efficiencies, similar to the findings in PC-12 cells. (FIG. 4). To demonstrate the specific contribution of the KSTQT motif in AAV-DMC1 in enhancing retrograde transport, the viruses were next applied selectively to the axons of the dorsal root ganglia, by culturing the cells in the Campenot format (Campenot, 1977. Proc. Natl. Acad. Sci. USA 74:4516-4519; Campenot, 1994. J. Neurobiol, 25:599-611). In these cultures, the cells were added to a central well partitioned with watertight barriers from separate chambers on either side. The neuron cell bodies remained isolated in the center well, but their axons and associated glia extended through the junctions into the side chambers due to a gradient of nerve growth factor (NGF). Axons and neuron cell bodies were thus sequestered in separate fluid environments (see FIG. 5A). The Campenot format serves as a stringent in vitro test for both efficient axonal uptake and retrograde transport, as a failure in either prevents successful gene transfer into the neurons.

Eight days after establishment, addition of the standard AAV-lacZ to selected side chambers failed to produce any lacZ expression in the central wells, in repeated trials. Exposure of axons to AAV capsids bearing either the KSTQT or HN motifs alone also produced either no or very few (2-4) lacZ-expressing cells in the corresponding central wells, although many cells in the treated side chambers expressed β-gal, particularly after exposure to AAV-DMC1. Only after chimeric AAV-HN1/DMC1 capsids were added to side chambers did significant numbers of central well cell bodies express β-gal (about 350 in one trial; see FIG. 5B). No clustering of β-gal positive neurons near the boundary with the treated side chamber was seen. Short pre-treatment of the side chambers with HN peptide prior to addition of the AAV-HN1/DMC1 effectively blocked lacZ expression in the central wells (FIG. 5C). Limited trials conducted with a different indicator, Red Fluorescent Protein (RFP) produced similar patterns.

That the expression pattern of the chimeric AAV-HN1/DMC1 vector in the Campenot cultures correlated with many more copies of its transgene reaching the central wells was confirmed by Real Time PCR. Heightened levels (1-2 logs) of transgene DNA were detected in central chambers 2 days after addition of the chimera to the axon chambers, compared with exposure to standard AAV-2—44.8×10³ copies versus 1.36×10³ respectively, in one trial. No virus signal was detected in culture medium from any of the central wells, or from side chambers not exposed to an rAAV, verifying the integrity of the watertight seals between the chambers, and confirming the appearance of the transgene and its protein product in neurons was not due to leakage of virus across the seals.

The results in the Campenot cultures also reflect several constraints upon standard rAAV-2 for gene transfer into neurons. In theory, the lack of transgene expression in the neuron cell bodies following axonal exposure to standard rAAV-2 could result from poor binding or uptake of the virus by the axons, inefficient retrograde transport, or both. Comparing the performance of the different mutant capsids against that of the standard rAAV-2 indicates both are important factors for the poor performance of the standard vector in this context. Simply providing a new affinity for a specific receptor on the axons via the HN motif was not sufficient to enhance vector efficiency. Conversely, even when the vector was altered to home to an efficient retrograde transport pathway, minimal neuronal transgene expression occurred in the absence of the other peptide conferring affinity for NMDA-R.

Gene Transfer into Other Cell Types Using Mutant AAV Capsids

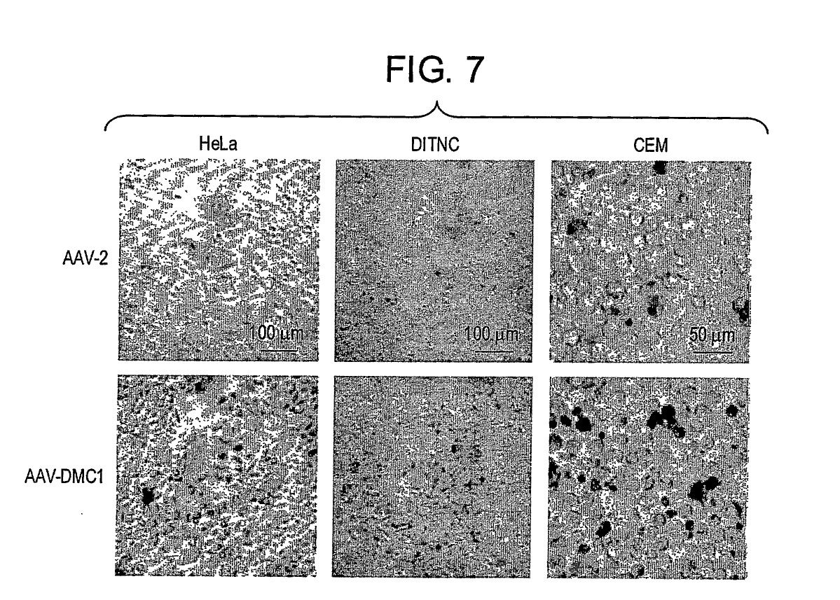

As noted in Campenot experiments, direct exposure to rAAV bearing the DMC1 motif produced more extensive transgene expression in side chamber cells exposed to these vectors than to standard rAAV-2. More glial cells were transduced by either the AAV-DMC1 or AAV-HN1/DMC1, compared with the standard rAAV-2 or AAV-HN1 vectors. The phenotypes of these capsids were then compared across a panel of non-neuronal cell lines, including 293, HeLa, CEM (a human T cell line), and DITNC (an immortalized rat astrocyte cell line). No enhancing effect of the HN peptide insert was observed in these cell types, in keeping with its specific affinity for NMDA receptors. In contrast, transgene expression after transduction with AAV-DMC1 was reproducibly higher in some lines than after exposure to standard AAV-2. Differences were marginal in CEMs, but more notable in others, ranging from less than 2 to more than 5 fold. Results from several trials are shown in FIG. 7.

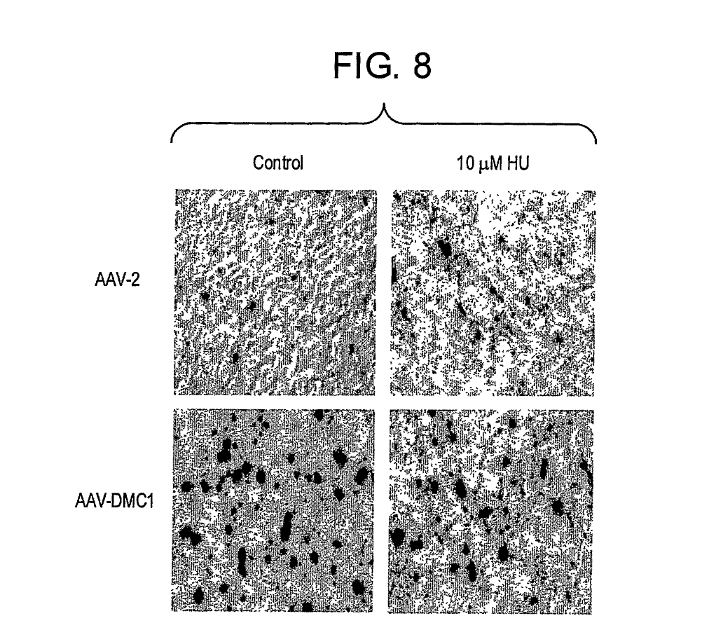

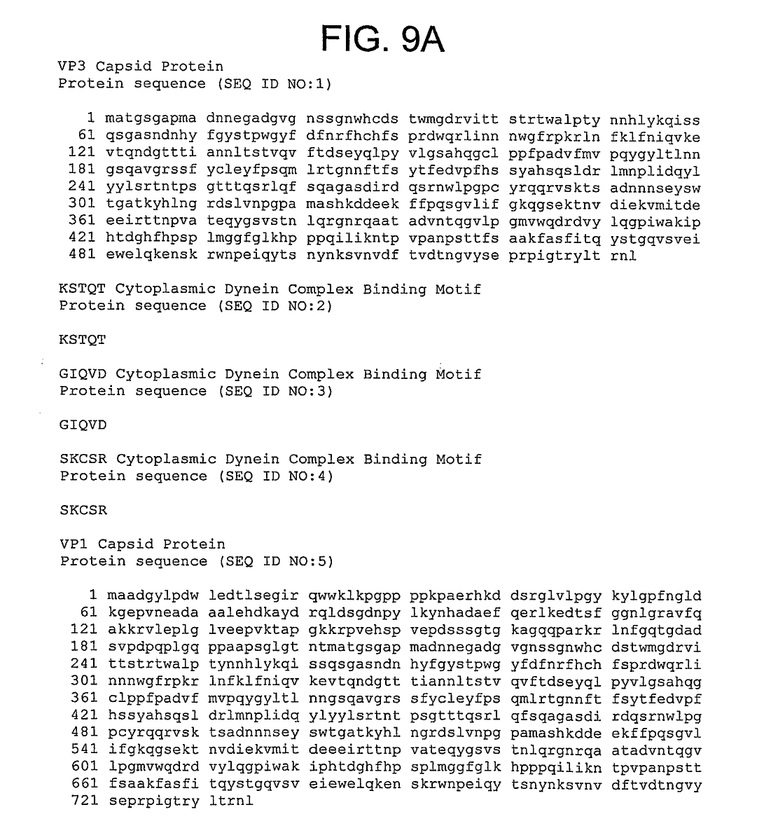

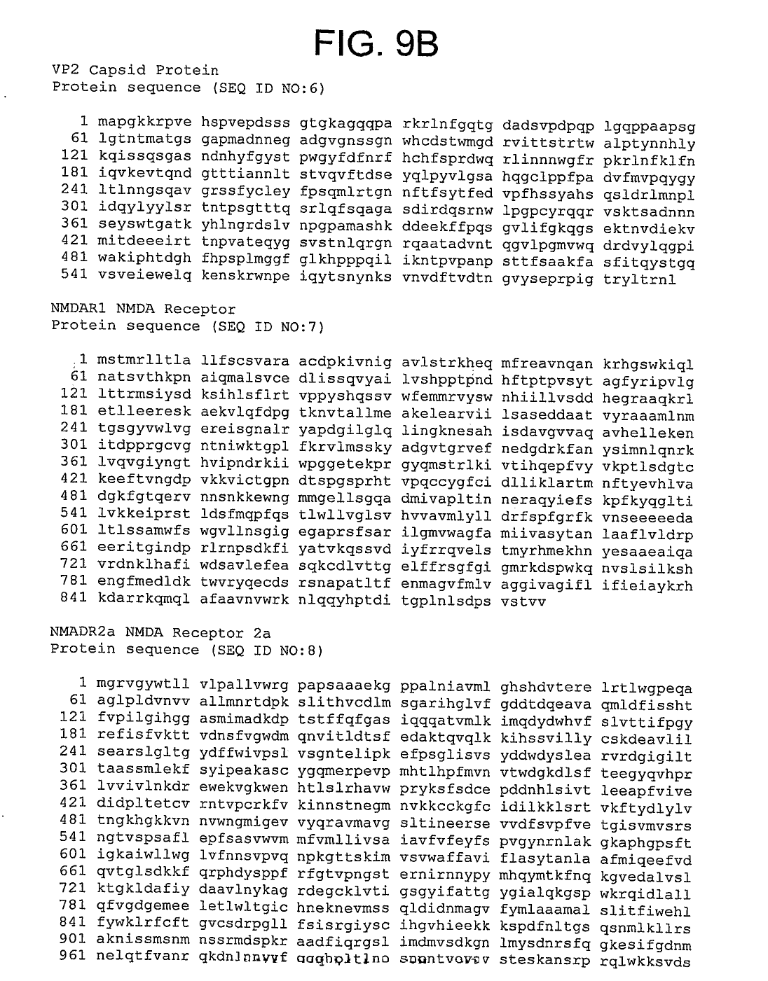

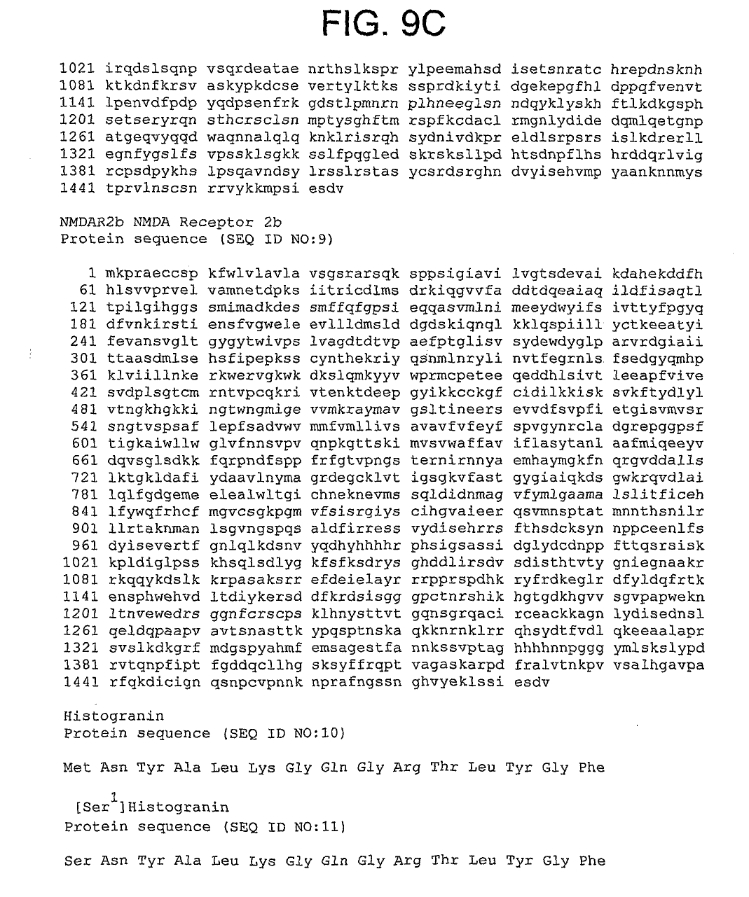

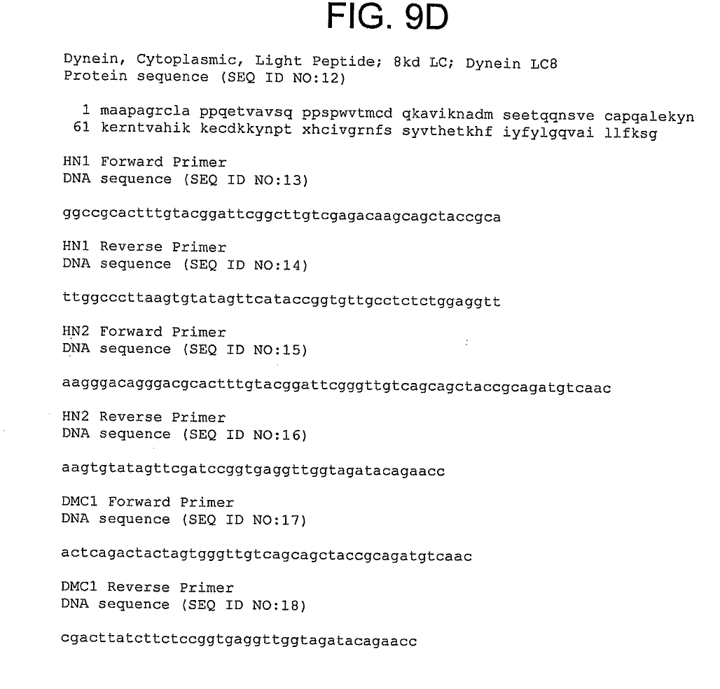

The retrograde transport pathway mediated by cytoplasmic dynein is active in cells other than neurons. To examine this effect of the DMC1 binding motif on transduction of non-neuronal cells more closely, AAV-DMC1 was tested on murine 3T3 cells, a cell type strongly resistant to standard AAV-2. Unlike cells that simply lack viral receptors, 3T3 cells are not impaired for binding or entry of AAV-2, yet still transduce very poorly. Instead, the rate-limiting step is impaired intracellular trafficking of the virus after entry due to impaired endosomal maturation (Hansen et al., 2000. J. Virol. 74:992-996; Hansen et al., 2001. J. Virol. 75:4080-4090). Virus particles remain localized primarily in early endosomes, from which they do not escape, and further maturation of the endosomes is impeded as compared to more easily transduced cell types such as 293. This blockage can be overcome by pretreatment of 3T3 cells with an agent such as hydroxyurea (HU) which promotes endosome acidification. Following HU treatment of the cells, AAV particles are then readily found in late endosomes and lysosomes from which escape is unimpaired. As the DMC1 capsid provides the virus with an alternative intracellular transport pathway, the susceptibility of 3T3 cells to gene transfer mediated by the mutant virus was compared against standard AAV in the presence or absence of HU. As shown in FIG. 8, the standard AAV-2 lacZ vector transduced the cells at only a very low level. Pretreatment with HU prior to transfection with the standard AAV-2 lacZ vector increased the number of cells expressing β-gal several fold. However, the percentage of cells expressing the transgene when it was delivered in the DMC1 capsid was much higher, even without HU treatment, and transduction was not enhanced further by HU.

The enhanced efficiency conferred by the DMC1 motif in other cell types is also interesting. Since this motif is not designed to affect entry, the findings indicate the alternative mode of intracellular transport active in this mutant functions more efficiently than the standard intracellular pathway traversed by the virus, at least in some cell types. In 3T3 cells, in which endosomal acidification is severely impaired, the presence of the DMC1 motif on the virus was required for successful transduction. This capability of the DMC1 motif suggests this class of mutation is valuable for gene therapy applications unrelated to the central nervous system. There have been recurring difficulties in practice with many strategies designed to target AAV, as well as other vectors, to heterologous receptors. In some cases this is due to an innate attribute of the targeted receptor, such as very slow uptake of ligand-receptor complexes. However, when a viral vector is redirected to a foreign receptor, after uptake some or most of the particles may traverse the intracellular pathway normally taken by its receptor-ligand complexes, which may be incompatible with expression of the genetic payload of the vector. This has been a problem with attempts to re-target viruses to, for example, the EGF receptor (Cosset et al., 1995. J. Virol. 69:6314-6322; Erlwein et al., 2002. Virology 302:333-341). By virtue of its ability to override some non-productive pathways, as in 3T3 cells and redirect the virus effectively to the DMC, motifs such as DMC1 can be beneficial to other targeting strategies.

### Example 4

### Binding of Mutant AAV Capsids to Cytoplasmic Dynein

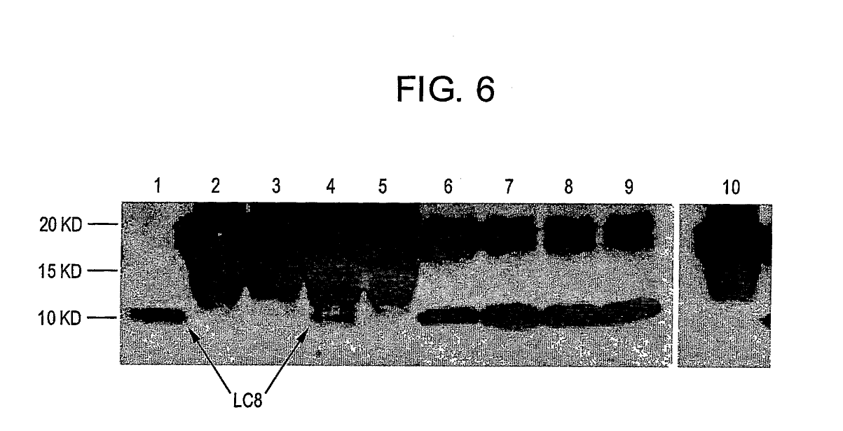

To verify the phenotypic changes in retrograde transport and transduction efficiency produced by the DMC1 peptide insert correlated with a new binding affinity of these capsids for a specific component of cytoplasmic dynein, plasmids encoding either the standard AAV-2 rep and cap gene sequences or the mutant bearing the DMC1 motif in cap were transfected into 293 cells. Cell lysates were prepared 24 hours later and immunoprecipitated under non-reducing conditions with an antibody against the AAV capsid protein. The immunoprecipitation products were then run on gels and visualized using a specific anti-serum against LC8, the cytoplasmic dynein light chain to which the KSTQT motif in DMC1 was designed to bind. Immunoprecipitation of the standard capsid did not co-immunoprecipitate LC8, but immunoprecipitation of the AAV-DMC1 capsid also brought down LC8, confirming binding of the mutant to this DMC polypeptide (FIG. 6).

### Example 5

### In Vivo Transduction of Chimeric AAV

As an initial test of efficacy in vivo, either the AAV-HN1/DMC1 mutant or regular rAAV-2 were applied to a standard animal model for retrograde transport. 10 μl of each vector were injected into the tongues of two groups of mice. Selected animals were sacrificed 24, 48, or 72 hours later. The tongues as well as brain stems were collected, and DNA was extracted for real time PCR. Samples of untransduced brain tissue from control mice were included as negative controls. Large quantities of vector DNA were present in tongues of animals receiving either vector, particularly immediately following injection. No signal above baseline was detectable in brain stems of animals receiving only standard rAAV-2 at any time point. Following injection of AAV-HN1/DMC1, no signal was present after 24 hours, but brainstems collected after 48 or 72 hours harbored up to several thousand copies of the vector transgene, as shown in Table 1.

TABLE 1
rAAV Copy Numbers in Treated Mouse Tongue and Brain Stems.

| | Brain stem² | | | Tongue³ | | |
|---|---|---|---|---|---|---|
| Vectors¹ | 24 h | 48 h | 72 h | 24 h | 48 h | 72 h |
| AAV-2 | 0 | 0 | 0 | 98,492 | 4,306 | 2,435 |
| AAVHN1/DMC1 | 0 | 490 | 2,771 | 146,944 | 18,078 | 677 |

¹10 μl of the indicated vector were injected into the front half of each tongue.
²20 mg of brain stem tissue including both hypoglossal nuclei from each mouse were dissected for DNA extraction at the indicated times.
³20 mg samples were also collected from each tongue near the injection site. Values reflect transgene copy numbers per tissue block. All values were subtracted from the mean of the negative control samples. Values less than 1 S.D. from the mean of the negative control were regarded as insignificant.

### Example 6

### Materials and Methods

The following materials and methods are used in the experiments described herein.

### Primary Cells and Cell Lines

293 cells, an adenovirus-transformed human embryonic kidney cell line, were maintained in Eagle's minimal essential medium (Mediatech Cellgro, Herndon, Va., USA) containing 10% fetal bovine serum (FBS) (Gibco BRL Life Technologies, Grand Island, N.Y., USA) and 100 U/ml penicillin-100 μg/ml streptomycin (pen-strep). HeLa, DITNC, and NIH 3T3 cells were cultured in Dulbecco's modified Eagle's medium (DMEM) (Gibco BRL) containing 10% FBS and pen-strep. PC-12 cells were maintained in RPMI 1640 (Cellgro) containing 10% heat-inactivated horse serum (Bibco BRL), 5% FBS and pen-strep. To induce differentiation, PC-12 cells were plated onto 24-well plates pre-coated with collagen at a starting density of 20,000 cells/well in maintenance medium overnight and were then cultured with 50 ng/ml NGF in RPMI 1640 containing 1% heat-inactivated horse serum and pen-strep for the next 7 days. NGF was replenished every 2 days. CEM cells were also cultured in RPMI 1640 with 10% FBS and pen-strep.

### Preparation of Campenot Cultures

Primary dorsal root ganglion (DRG) neurons from the superior cervical ganglia of newborn Sprague Dawley rats were isolated as described previously (Heerssen et al., 2004. Nat. Neurosci. 7:596-604). Campenot cultures were established from some preparations of DRG as described by Campenot (1977. Proc. Natl. Acad. Sci. USA 74:4516-4519 and 1994. J. Neurobiol. 25:599-611). Briefly, after isolation, about 10⁵ neurons were plated in central compartments formed by Teflon dividers placed across parallel scratches made on 35 mm dishes. The central compartments contained DMEM with penstrep, and 100 nM AraC supplemented with 10 ng/ml nerve growth factor (NGF), while the side compartments contained the same medium supplemented with 100 ng/ml NGF. This gradient of NGF across the 2 compartments guided the growth of neurites from the central chambers into the side compartment along the scratches. On the sixth day of culture, the concentration of NGF in the central compartment was further reduced to 1 ng/ml. The cultures were used for experiments on Day 8. Survival at this stage was estimated at 50 percent of the original cells.

### Detection of β-Galactosidase

Expression of β-gal, was assessed by X-gal staining as described previously (Madry et al., 2003. Hum. Gene Ther. 14:393-402). Briefly, cells were fixed with 2% formaldehyde and 0.2% gluteraldehyde in PBS (pH 7.6) at 4° C. for 5 min followed by 3 washes with PBS. The cells were then incubated with 1 mg/ml X-gal, 1.64 mg/ml potassium ferricyanide, 2.12 mg/ml potassium ferrocyanide, 2 mM magnesium chloride in PBS (pH 7.6) at 37° C. for 12 hours.

DNA Extraction from Tissue and Real Time PCR

Male, 5 week old Balb/C mice were anaesthetized by i.p. injection of 100 mg/kg of ketamine/xylazine (1:1). Approximately 10 μl of standard or mutant AAV vector were injected in the front half of the tongue. The mice were allowed free access to food and water after recovering from anesthesia. At 24, 48, or 72 hours after tongue injection, mice from each group received an i.p. overdose injection of pentobarbital. A 20±2 mg tissue block was quickly dissected from the tongue around the injection site, and the brain stem containing both sides of the hypoglossal nuclei was collected from each mouse. Samples of cerebral cortex were collected from other mice as negative controls. DNA was extracted using the QIAamp DNA Mini Kit (QIAGEN Inc., Valencia, Calif.). The copy number of the vector transgene in each sample was assayed by real-time PCR using a primer and probe set and cycling conditions previously described (Lewis et al., 2002. J. Virol. 76:8769-8775).

### Western Blotting and Immunoprecipitation

293 cells (6-7×10⁶ per 100 mm dish) were transfected with 10 μg of each AAV vector plasmid using a calcium phosphate precipitation method standard in the art. Twenty four hours after transfection, the cells were scraped on ice into phosphate buffered saline (pH 7.4) containing 1% Igepal CA-630, 0.5% sodium deoxycholate, 0.1% sodium dodecyl sulfate, 1 mg/ml leupeptin, 1 mg/ml pepstatin, 1 mM benzamidine, 1 mM sodium orthovanadate, 1 mM phenylmethylsulphonyl fluoride, 200 nM staurosporine, and 3.3 U/ml apyrase. The cell suspensions were homogenized using a 1 ml Wheaton homogenizer for 10 strokes and centrifuged at 10,000 g, 4° C. for 15 min. Control cell lysate was prepared in the same way from untransfected cells. The clarified lysates were then used for immunoprecipitation. The lysates were pre-cleared with control rabbit antiserum and protein G (Santa Cruz Biotechnology, Inc., Santa Cruz, Calif.) and then incubated with a mouse monoclonal antibody against an epitope of the AAV-2 capsid, A20 (American Research Products, Inc., Belmont, Mass.), together with protein G on a rocker at overnight 4° C. The immunocomplexes were then pelleted by centrifugation at 2,500 rpm for 30 seconds, washed with PBS, and repelleted 3 times. The pellets were then run on sodium dodecyl sulfate polyacrilimide gel electrophoresis (SDS-PAGE), and immunoblotted with a rabbit polyclonal antibody against LC8 to check for co-precipitation of this protein.

All patents, patent applications including U.S. provisional application No. 60/676,032, and publications mentioned in this specification are herein incorporated by reference to the same extent as if each independent patent, patent application, or publication was specifically and individually indicated to be incorporated by reference.

What is claimed is:

1. A modified capsid protein comprising a modification that increases binding of a viral particle comprising said capsid protein to an NMDA receptor, relative to the binding of a viral particle not comprising said capsid protein, wherein said modification is sufficient to increase the binding of said viral particle comprising said capsid protein to a neuron comprising said NMDA receptor.

2. The modified capsid protein of claim 1, wherein said modification comprises an insertion of histogranin, or a fragment thereof.

3. The modified capsid protein of claim 2, wherein said capsid protein is an AAV capsid protein.

4. The modified capsid protein of claim 3, wherein said capsid protein is a VP3 capsid protein.

5. The modified capsid protein of claim 4, wherein said insertion is between amino acids 583 and 590 of said VP3 capsid protein.

6. A viral vector comprising the modified capsid protein of claim 1.

7. The modified capsid protein of claim 1, wherein said capsid protein further comprises a deletion of amino acid sequence from said capsid protein.

8. The modified capsid protein of claim 7, wherein said capsid protein is an AAV capsid protein and said deletion comprises a deletion of residues 584-589 of the VP3 capsid protein.

9. The modified capsid protein of claim 1, wherein said capsid protein further comprises a modification that substantially decreases binding of said viral particle comprising said capsid protein to a heparin sulfate proteoglycan, relative to the binding of a viral particle not comprising said capsid protein to said heparin sulfate proteoglycan.

10. The modified capsid protein of claim 9, wherein said capsid protein comprises at least 5% of capsid proteins present in said viral particle.

11. The modified capsid protein of claim 9, wherein said capsid protein is an AAV capsid protein and comprises a mutation or deletion of one or more of the following amino acid residues of a VP3 capsid protein: R484, R487, R585, R588, and K532.

12. A polynucleotide encoding the capsid protein of claim 1.

13. A vector comprising the polynucleotide of claim 12.

14. The vector of claim 13, wherein said vector is a viral vector.

15. The vector of claim 14, wherein said viral vector is an AAV vector.

16. A modified capsid protein comprising a modification that increases binding of a viral particle comprising said capsid protein to a component of the cytoplasmic dynein complex, relative to the binding of a viral particle not comprising said capsid protein, wherein said modification is sufficient to enhance retrograde transport of a particle comprising said capsid protein.

17. The modified capsid protein of claim 16, wherein said modification comprises an insertion of a cytoplasmic dynein complex binding motif into said capsid protein.

18. The modified capsid protein of claim 17, wherein said cytoplasmic dynein complex binding motif is selected from the group consisting of KSTQT (SEQ ID NO:2), GIQVD (SEQ ID NO:3), and SKCSR (SEQ ID NO:4).

19. The modified capsid protein of claim 16, wherein said capsid protein further comprises a modification that substantially decreases binding of a viral particle comprising said capsid protein to a heparin sulfate proteoglycan, relative to the binding of a viral particle not comprising said capsid protein to said heparin sulfate proteoglycan, wherein said modification is sufficient to decrease binding of a viral particle comprising said capsid protein to a cell comprising said heparin sulfate proteoglycan when said capsid protein comprises at least 5% of capsid proteins present in said viral particle.

20. The modified capsid protein of claim 19, wherein said capsid protein is an AAV capsid protein.

21. The AAV capsid protein of claim 20, wherein said capsid protein comprises a mutation or deletion of one or more of the following amino acid residues of a VP3 capsid protein: R484, R487, R585, R588, and K532.

22. A polynucleotide encoding the capsid protein of claim 16.

23. A viral vector comprising the polynucleotide of claim 22.

24. The viral vector of claim 23, wherein said viral vector is an AAV viral vector.

25. A chimeric viral particle with (i) increased binding to a neuron, said neuron comprising an NMDA receptor, and (ii) enhanced retrograde transport along a neuronal axon, said viral particle comprising at least two modified capsid proteins, each of said capsid proteins comprising one of (1) a modification that increases binding of said viral particle to an NMDA receptor, and (2) a modification that increases binding of said viral particle to the cytoplasmic dynein complex, wherein said viral particle has at least one of (a) increased binding to a neuron comprising an NMDA receptor and (b) increased retrograde transport when said viral particle contacts a neuron.

26. The chimeric viral particle of claim 25, wherein said capsid protein having a modification that increases binding of a viral particle comprising said capsid protein to said NMDA receptor, wherein said modification comprises an insertion of histogranin, or a fragment thereof, into said capsid protein.

27. The chimeric particle of claim 25, wherein said capsid protein having a modification that increases binding of said particle to said cytoplasmic dynein complex comprises an insertion of a cytoplamsic dynein binding motif.

28. The chimeric particle of claim 27, wherein said insertion comprises an amino acid sequence selected from the group consisting of KSTQT (SEQ ID NO:2), GIQVD (SEQ ID NO:3), and SKCSR (SEQ ID NO:4).

29. The chimeric viral particle of claim 25, wherein said viral particle is an AAV particle.

30. A method of introducing a nucleic acid into a neuron, said method comprising administration of a vector comprising a modified capsid protein, said capsid protein comprising a modification that increases binding to an NMDA receptor relative to the binding of a capsid protein lacking said modification to said NMDA receptor, wherein said modification is sufficient to increase the binding of a vector comprising said capsid protein to a neuron comprising said NMDA receptor.

31. The method of claim 30, wherein said neuron is in a subject.

32. The method of claim 31, wherein said subject is a human.

33. The method of claim 31, wherein said method allows enhanced expression of a nucleic acid in a neuron of said subject relative to a vector lacking said capsid protein.

34. A method of introducing a nucleic acid into a cell, said method comprising administration of a vector comprising a modified capsid protein, said capsid protein comprising a modification that increases binding of said capsid protein to the cytoplasmic dynein complex relative to the binding of a capsid protein lacking said modification to said cytoplasmic dynein complex, wherein said modification is sufficient to enhance retrograde transport in a cell of a viral vector comprising said capsid protein.

35. The method of claim 34, wherein said cell is in a subject.

36. The method of claim 35, wherein said subject is a human.

37. The method of claim 35, wherein said method allows enhanced expression of said nucleic acid in a cell of said subject relative to a vector lacking said capsid protein.
